(19) United States
(12) Patent Application Publication
(10) Pub. No.: US 2009/0204140 A1
(43) Pub. Date: Aug. 13, 2009

(54) Surgical trocar

(76) Inventors: John E. Dandl, Chico, CA (US); Donald P. Suderman, Chico, CA (US)

Correspondence Address: Robert Charles Hill, 235 Montgomery Street #821, San Francisco, CA, 94104, US

(21) Appl. No.: 12/069,782

(22) Filed: Feb. 13, 2008

(51) Int. Cl. A61B 17/34; A61M 27/00

(52) U.S. Cl. 606/185; 604/541

(57) ABSTRACT

A surgical trocar includes a trocar needle with a sharp tip for puncturing and passing through the skin of a patient. A hose barb disposed on a distal end of the trocar needle retains a drain tubing while they are being threaded through a skin puncture. A head formed aft of the tip of the trocar needle has a locking ridge to provide a grip. A tapered section of the trocar needle is disposed aft of the head and has its minimum diameter adjacent to the locking ridge, and its maximum diameter in the direction of the hose barb. Thus, a gradually increasing diameter of the tapered section aft of the head substantially reduces any snagging or tearing of a patient's skin around an associated skin puncture during use.

### BACKGROUND OF THE INVENTION

1. Field of the Invention

The present invention relates to surgical trocars, and in particular to trocars designed to puncture and pass through skin and provide a drain after surgery without snagging or tearing the skin around the puncture.

2. Description of Related Art

Wikipedia defines a trocar as a hollow cylinder with a sharply pointed end, often three-sided, that is used to introduce cannulas and other similar implements into blood vessels or body cavities. Trocars are also used as ports in laparoscopic surgery.

The trocar is often passed inside a cannula, and functions as a portal for the subsequent placement of other devices, such as a chest drain, intravenous cannula, etc. Surgical trocars are used for laparoscopic surgery. They can be a means of introduction for laparoscopic hand instruments, e.g., scissors and graspers, to do surgery previously done by making a large abdominal incision. Surgical trocars are usually a single-patient-use instrument, and have graduated from three point designs to flat bladed dilating-tips, or ones that are entirely blade free.

Trocars are also used near the end of the embalming process to provide drainage of bodily fluids and organs after the vascular replacement of blood with embalming chemicals. It is attached to a suction hose, in a process of removing organs known as aspiration.

Surgical trocars resemble large, very sharp, needles with surgical tubing attached to the back end. The tubing is perforated to promote drainage along its distal length. The surgeon forces the point of the trocar out from inside a wound near the opening that will be sutured later. The sharp end of the trocar is too dangerous and difficult to pull out through the skin directly, so conventional products include a sheath inside a handle that will receive and lock on to the trocar's sharp tip. This then protects the surgeon and all others from being poked or jabbed by the bloody tip, and provides a large handle for the surgeon to tug on. See, FIGS. 8A-8C in U.S. Pat. No. 6,613,039, issued to Robert S. Namba, and paragraphs [0173] and [0179] of Publication No. US 2004/0092891, published May 13, 2004.

Prior art trocars invariably include a groove around the tip for the sheath and handle to lock onto. But such grooves will often snag the skin of the patient and tear.

### SUMMARY OF THE INVENTION

Briefly, a surgical trocar embodiment of the present invention includes a trocar needle with a sharp tip for puncturing and passing through the skin of a patient. A hose barb disposed on a distal end of the trocar needle retains a drain tubing while they are being threaded through a skin puncture. A head formed aft of the tip of the trocar needle has a locking ridge that provides a grip. A tapered section of the trocar needle is disposed aft of the head and has its minimum diameter adjacent to the locking ridge, and its maximum diameter in the direction of the hose barb.

An advantage of the present invention is that a surgical trocar is provided that substantially reduces any snagging or tearing of a patient's skin around an associated skin puncture during use.

Another advantage of the present invention is that a trocar is provided that is safe for the surgeon and others to use.

The above and still further objects, features, and advantages of the present invention will become apparent upon consideration of the following detailed description of specific embodiments thereof, especially when taken in conjunction with the accompanying drawings.

### BRIEF DESCRIPTION OF THE DRAWINGS

FIG. 1A is a cross-section and perspective view diagram of a trocar system embodiment of the present invention showing the trocar needle, drain tubing, receiver base, receiver guard, and receiver handle all disassembled;

FIG. 1B is a cross-section and perspective view diagram of the trocar system of FIG. 1A showing the trocar needle assembled to the drain tubing, and the handle, guard, and base are assembled to form the trocar receiver before their being used on a patient; and

FIG. 1C is a cross-section and perspective view diagram of the trocar system of FIGS. 1A and 1B showing the trocar needle and drain tubing after the tip has been pushed through the skin of a patient, and the assembled trocar receiver has locked on to the trocar needle to allow the surgeon to pull the trocar needle and drain tubing through the skin puncture.

### DETAILED DESCRIPTION OF THE INVENTION

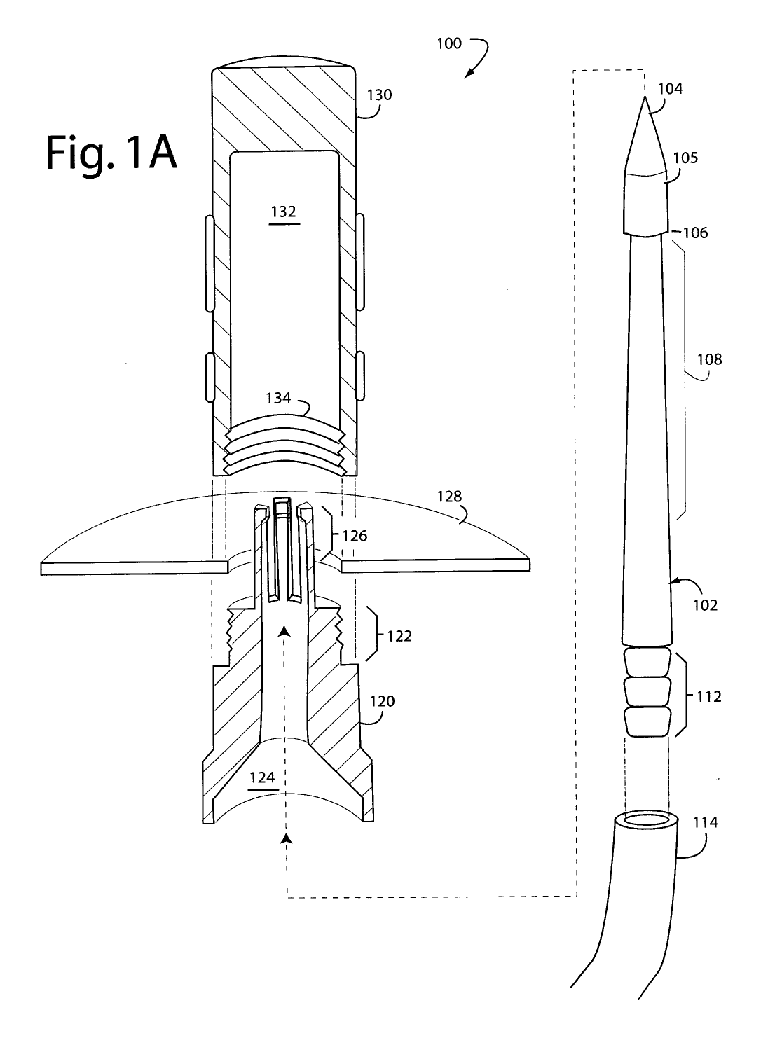

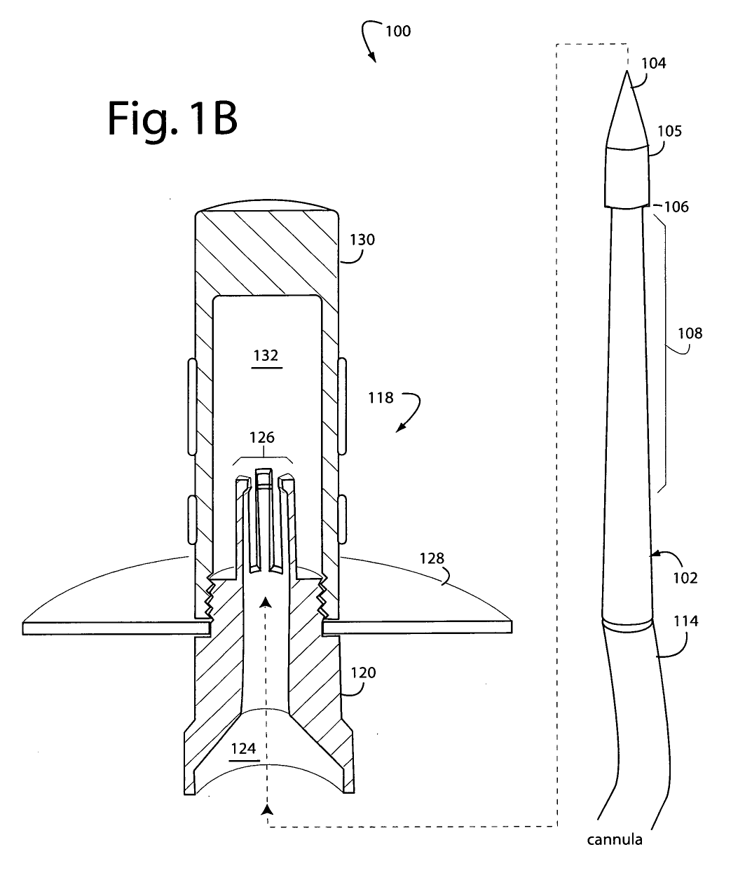

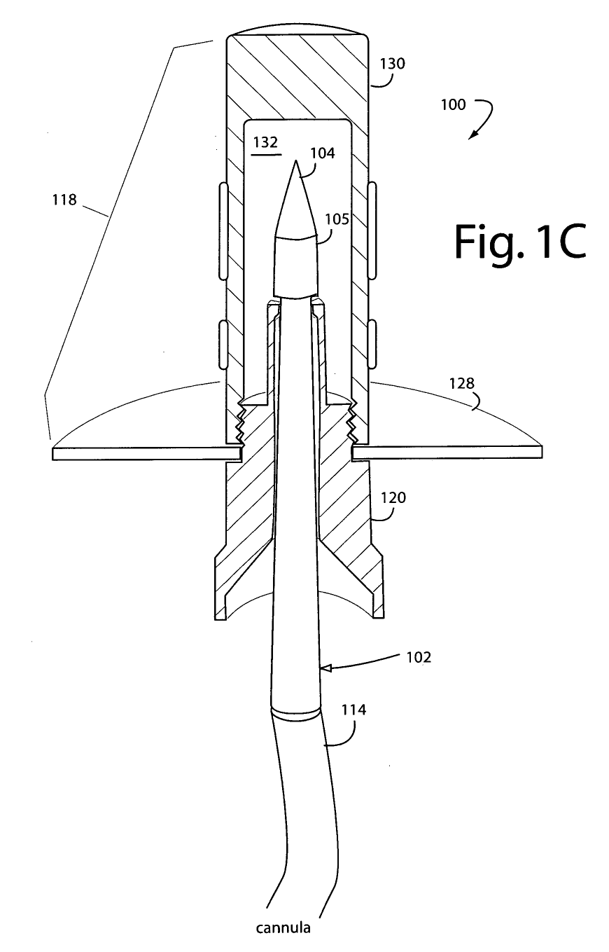

FIGS. 1A-1C represent a surgical trocar system embodiment of the present invention, and is referred to herein by the general reference numeral 100. Trocar system 100 comprises a straight or slightly curved trocar needle 102 with a sharp tip 104 that forms a head 105 with a ridge 106. A tapered section 108 gradually increases back in diameter to the size of the head 105 in the direction of a hose barb 112. In one embodiment, such tapered section was 3-mm in diameter increasing to 5-mm in diameter over a distance of 40-mm. The trocar needle was 150-mm in overall length. A drain tubing 114 provides a way to drain a surgical site after closure and suturing.

The tapered section 108 is important in that there is no groove formed aft of the ridge 106 that can snag and tear the patient's skin when the needle 102 is being pushed through by a surgeon.

A trocar receiver 118 (FIG. 1C) is assembled from three parts that screw together. A base 120 includes a threaded collar 122, and a funnel 124 that receives and guides the tip 104 of trocar needle 102 after puncturing the skin during the installation of the cannula. Locking fingers 126 are spread by the tip 104 and will lock behind ridge 106. A round guard 128 protects the hands of the surgeon from being poked accidently by tip 104. A handle 130 with a hollow cavity 132 and female thread 134 screws to base 120.

In one embodiment, the round guard 128 was 45-mm in outside diameter, had a 12-mm hole in the center like a sheet-metal fender washer, and was 1-mm thick aluminum. The handle 130 was diamond-pattern knurled aluminum, 18-mm in diameter, and 85-mm long.

The trocar receiver 118 will essentially lock permanently onto the trocar needle 102, and they both are disposed of after a single use. The handle 130 will keep the sharp tip 104 safe inside so as not to create a sharps hazard that is already a bio-hazard and contaminated by blood, tissues, and fluids after use.

In use, the parts of trocar system 100 will probably arrive disassembled, as in FIG. 1A. The trocar needle 102, drain tubing 114, receiver base 120, receiver guard 128, and receiver handle 130 are used once in only one patient, and discarded after use. FIG. 1B shows the trocar needle 102 assembled to the drain tubing 114, and the assembled trocar receiver 118 before being used on a patient. The tip 104 and head 105 of the trocar needle 102 is pushed by the surgeon through the skin of a patient. FIG. 1C shows the assembled trocar receiver 118 locked on to the trocar needle 102. Trocar receiver 118 provides a good, safe grip for the surgeon to pull the trocar needle 102 and drain tubing 114 through the skin puncture. The drain tubing 114 is left behind in the patient's skin to drain the site, and the rest are discarded.

Although particular embodiments of the present invention have been described and illustrated, such was not intended to limit the invention. Modifications and changes will no doubt become apparent to those skilled in the art, and it was intended that the invention only be limited by the scope of the appended claims.

The invention claimed is:

1. A surgical trocar, comprising:
a trocar needle with a sharp tip for puncturing and passing through the skin of a patient;
a hose barb disposed on a distal end of the trocar needle for retaining a drain tubing while they are being threaded through a skin puncture;
a head formed aft of said tip of the trocar needle and having a locking ridge that provides a grip;
a tapered section of the trocar needle disposed aft of the head that has its minimum diameter adjacent to said locking ridge, and its maximum diameter in the direction of the hose barb;
wherein, a gradually increasing diameter of the tapered section aft of the head substantially reduces any snagging or tearing of a patient's skin around an associated skin puncture during use.

2. The surgical trocar of claim 1, further comprising:
a drain tubing for attachment to the hose barb and that is passed with the trocar needle through said associated skin puncture during use.

3. The surgical trocar of claim 2, further comprising:
a receiver providing for the capture of the trocar needle and a lock mechanism to engage the head and its locking ridge;
wherein, a surgeon is provided a device to pull the trocar needle and drain tubing through said associated skin puncture during use.

4. The surgical trocar of claim 3, further comprising:
a receiver handle forming a part of the receiver, and providing a grip for said surgeon to pull the trocar needle and drain tubing through said associated skin puncture during use, and further providing a hollow cavity over the head and sharp tip of the trocar needle to make disposal of the whole safer.

5. The surgical trocar of claim 3, further comprising:
a receiver base forming a part of the receiver, and including a funnel to guide the head of the trocar needle up and into said lock mechanism, and further providing a safe way for the surgeon to capture the head and sharp tip of the trocar needle for threading out.

6. The surgical trocar of claim 3, further comprising:
a receiver guard forming a part of the receiver, and providing a safe way for the surgeon to capture the head and sharp tip of the trocar needle for threading out.

7. A surgical trocar system, comprising:
a trocar needle with a sharp tip for puncturing and passing through the skin of a patient;
a hose barb disposed on a distal end of the trocar needle for retaining a drain tubing while they are being threaded through a skin puncture;
a head formed aft of said tip of the trocar needle and having a locking ridge that provides a grip;
a tapered section of the trocar needle disposed aft of the head that has its minimum diameter adjacent to said locking ridge, and its maximum diameter in the direction of the hose barb;
a drain tubing for attachment to the hose barb and that is passed with the trocar needle through said associated skin puncture during use; and
a receiver providing for the capture of the trocar needle and a lock mechanism to engage the head and its locking ridge, including:
a receiver handle forming a part of the receiver, and providing a grip for said surgeon to pull the trocar needle and drain tubing through said associated skin puncture during use, and further providing a hollow cavity over the head and sharp tip of the trocar needle to make disposal of the whole safer; and
a receiver base forming a part of the receiver, and including a funnel to guide the head of the trocar needle up and into said lock mechanism, and further providing a safe way for the surgeon to capture the head and sharp tip of the trocar needle for threading out;
a receiver guard forming a part of the receiver, and providing a safe way for the surgeon to capture the head and sharp tip of the trocar needle for threading out.
wherein, a surgeon is provided a device to pull the trocar needle and drain tubing through said associated skin puncture during use; and
wherein, a gradually increasing diameter of the tapered section after of the head substantially reduces any snagging or tearing of a patient's skin around an associated skin puncture during use.

8. A method for reducing any snagging or tearing of a patient's skin around an associated skin puncture using a trocar, comprising:
providing a head on a tip of a trocar needle with a locking ridge; and
providing a tapered section on said a trocar needle just aft of said locking ridge;
wherein, a gradually increasing diameter of the tapered section after of the head substantially reduces any snagging or tearing of a patient's skin around an associated skin puncture during its one and only use.

9. The method of claim 8, further comprising:
using a receiver which can capture said trocar needle with a lock mechanism to engage said head and its locking ridge.
